(19) United States
(12) Patent Application Publication
(10) Pub. No.: US 2025/0213375 A1
(43) Pub. Date: Jul. 3, 2025

(54) A STENT THAT REDUCES DAMAGE AFTER BRAIN HAEMORRHAGE

(71) Applicant: BAHCESEHIR UNIVERSITESI, Besiktas/Istanbul (TR)

(72) Inventors: Bora BUYUKSARAC, BESIKTAS / ISTANBUL (TR); Kutay CANTASIR, BESIKTAS / ISTANBUL (TR); Mustafa Can KURT, SARIYER / ISTANBUL (TR)

(73) Assignee: BAHCESEHIR UNIVERSITESI, Besiktas/Istanbul (TR)

(21) Appl. No.: 18/851,402

(22) PCT Filed: Dec. 22, 2023
(86) PCT No.: PCT/TR2023/051653

§ 371 Date: Sep. 26, 2024

(30) Foreign Application Priority Data
Dec. 27, 2022 (TR) 2022/020723

(51) Int. Cl. A61F 2/90

(52) U.S. Cl. CPC A61F 2/90; A61F 2230/0067; A61F 2250/0039

(57) ABSTRACT

The invention relates to a stent that reduces the damage that occurs after brain haemorrhage, for use in the treatment of aneurysms that have ruptured or require intervention. The stent of the invention comprises an exoskeleton (1), a curved conical structure (2) positioned in the centre of the stent (1), bridge (3), flap (4) and silicone sheath (5).

### TECHNICAL FIELD OF THE INVENTION

The invention relates to a stent that comprises a curved conical structure (2) that regulates blood flow within the aneurysm and reduces damage after brain haemorrhage, for use in the treatment of aneurysms that are ruptured or require intervention.

### STATE OF THE ART

The weakening and enlargement of the aorta and brain vessel walls due to some reasons and the formation of bubbles in these areas are called aneurysm. Aneurysms, which are generally seen in the brain and aortic vessels, can also occur in different parts of the body. Bubbles, which do not cause any symptoms in most patients, are generally not dangerous; however, aneurysm ruptures occurring in the brain and main arteries cause cerebral haemorrhage and internal bleeding. Aneurysms may be due to congenital vascular maldevelopment, but they may also develop after high blood pressure, arteriosclerosis, infections (inflammation of the vessel) or trauma [1]. Aneurysms are usually located at the base of the brain and cause bleeding in the cerebrospinal fluid there. The annual bleeding risk of aneurysms is approximately 1%. Aneurysm types are classified depending on the region in the vessel wall where they develop and the way they develop. Aneurysms are classified as saccular (sac-shaped) and fusiform (spindle-shaped) according to their form of development.

Saccular aneurysm, a type of brain aneurysm, is the most common type of brain aneurysm that appears as a sac on the brain vessels and accounts for approximately 90 percent of all brain aneurysms. Saccular aneurysm is usually seen in the area where five large blood vessels in the brain meet, called the centre of the brain, known in medicine as the Willis polygon, and in the branches of these vessels. Arterial pressure can cause the weakened aneurysm wall to rupture over time. When a saccular aneurysm ruptures, blood from the artery jumps to the brain. A ruptured aneurysm is a serious condition that requires emergency medical treatment. Fusiform aneurysm, another type of brain aneurysm, appears as a spindle-shaped expansion involving a long section of the vessel. These types of aneurysms may rupture and bleed, expand excessively and cause pressure on the surrounding brain tissue, or cause stroke-like symptoms when clots develop, and detachable residues cause blockage (embolism) in normal brain vessels [2].

A brain aneurysm can leak or rupture, causing bleeding in the brain (haemorrhagic stroke). Acute bleeding from an aneurysm can physically damage the brain and lead to acute transient global cerebral ischaemia. Transient global cerebral ischaemia secondary to increased intracranial pressure may also trigger sympathetic nervous system activation, leading to systemic complications. Most often, brain aneurysm rupture occurs in the space between the brain and the thin tissues covering the brain. This type of haemorrhagic stroke is called subarachnoid haemorrhage (SAH). Even though SAH accounts for only 5% of all strokes, the case fatality rate is 40% due to delayed cerebral ischaemia and neurological problems that occur days after bleeding. Additionally, many survivors of SAH have permanent neurological, cognitive, or functional problems [3].

Details of the cellular pathophysiology have recently begun to be explained. Transient global cerebral ischaemia, whose contribution mechanism has not yet been fully revealed, and blood in the subarachnoid space cause microcirculation disorders, microthrombosis, disruption of the blood-brain barrier, cytotoxic and vasogenic cerebral oedema, and neuronal and endothelial (vascular wall cell) cell death. As a result, early brain damage is observed [4]. Following the initial haemorrhage, delayed cerebral ischaemia occurs within 3-14 days, which is associated with mechanisms such as cerebral vascular dysfunction, microthrombosis, cortical spreading depolarisations, neuroinflammation, and other mechanisms of early brain damage. Delayed cerebral ischaemia plays an important role in the morbidity of SAH. Various evaluation methods are used in the clinic, taking into account meningismus and other neurological conditions due to the amount of subarachnoid blood that plays a role both mechanically and biochemically in the development of all these processes, the rate of removal of blood from the subarachnoid space, and the inflammation caused by the blood in the subarachnoid space [5].

Clip placement on the aneurysm neck and endovascular coil embolisation are the treatment methods used. Among the endovascular methods that continue to develop are stent-assisted coiling, balloon-assisted coiling, flow-directing stents, and flow-disrupting technologies. The main goal of current treatments is to cut off blood flow to the aneurysm and prevent it from rupturing or bleeding again. However, there is no treatment to remove subarachnoid blood due to ruptured aneurysm, which affects the clinical course of the patient in the long term. Although subarachnoid blood is tried to be removed from the environment while surgically attaching the clip, the clinically expected improvements cannot be achieved from these interventions, both because the haematoma has already organised and the mediators of early and late brain damage have been released into the environment, and because it has already shown its mechanical effects in the early phase. In various clinical studies, findings regarding the clinical improvement achieved by removing subarachnoid blood during surgery vary. Depending on the appropriate technique and the surgeon's experience, satisfactory results can be achieved by removing the clot from the subarachnoid space as early as, and in as much quantity as possible [6]. Additionally, there is data showing that treatments that attempt to remove blood from the subarachnoid space by intrathecal-cisternal fibrinolysis are beneficial [7]. However, in a study, an increase in morbidity due to vasospasm was observed in patients with thick and widespread subarachnoid haemorrhage (≥4 mm thickness and haematoma in ≥3 basal cisterns) on computed tomography scans at the time of first admission to the hospital, an increase in all-cause mortality was observed in the 6-week period after bleeding, and an association with poor clinical outcomes was found at follow-ups 12 weeks after bleeding [8]. In addition to the importance of clot removal, the fact that irreversible brain damage due to aneurysm rupture occurs immediately after haemorrhage indicates that clot removal should be done as early as possible [9]. In addition, it is very important to maintain blood circulation distal to the aneurysm (the area after the arterial blood circulation passes the aneurysm neck) to reduce ischaemic damage to the general brain [10].

Recent studies have shown that in aneurysmal subarachnoid haemorrhages, metabolites formed during direct contact of blood with the brain tissue and its removal from the subarachnoid area may have an impact on the clinical course of the disease. Among the existing endovascular treatment methods, flow diverting stents, coil embolisation and WEB (Woven Endobridge) embolisation system reduce the blood flow in the aneurysm and cause it to thrombose, and as a result, the aneurysm remains out of the arterial circulation. Clipping, a different treatment method, aims to close the aneurysm neck and remove the bleeding focus from the circulation. WEB is actually a type of intrasaccular flow disruptor device. This device, which has a mesh-like physical structure, is delivered to the cerebral circulation via an endovascular approach, placed inside the intracranial aneurysm sac, and covers the neck of the intracranial aneurysm to prevent blood from entering the sac. However, since the main purpose of these methods is to stop bleeding, these methods cannot remove the blood in the subarachnoid space [11]. In the state of the art, there are various studies on the feasibility of flow diverting stents for intervention in the acute period [12]. Said flow directing stents work by shaping the flow of blood within the vessel, reducing the pressure applied to the vessel wall and the amount of blood entering the aneurysm. However, flow diverting stents cannot completely cut off the flow into the aneurysm sac and, more importantly, cannot remove blood from the subarachnoid space [13].

The patent application numbered WO2022022143A1 in the state of the art relates to a stent comprising a membrane in the middle part. Here, there is a membrane structure inside the stent body, and the proximal end of this membrane structure is fixed to the stent body. In one embodiment of the invention, the cross-section of the pipe (membrane) through which the flow is provided is designed in different sizes, as shown in FIG. 7. Thus, the flow in the narrow section of the pipe is faster and the flow in the wide section is slower. According to the Bernoulli equation, the increase in flow rate is accompanied by a decrease in liquid pressure, so the pressure in the pipe is less in the narrow section and more in the wide section. It is stated that this membrane-containing stent is used in blood vessels with aneurysms.

Since the invention described in said patent document tries to provide the required lumen area narrowing rate with a straight conical narrowing, turbulence and water hammer will occur at the narrowing outflow section. Since sufficient vacuum cannot be created within the vessel, the stent will not be able to reabsorb blood from the subarachnoid area [14]. In order to avoid the effects of turbulent flow and water hammer, the lumen narrowing angle of the membrane structure must be reduced. In this case, a longer membrane structure will be needed to create a narrowing of the lumen area that can reabsorb blood from the subarachnoid space into the vessel. However, in this case, the length of the stent structure will be too long, and it will be difficult to place the stent in tortuous brain arteries. In addition, since the membrane structure of the stent will be affected by the convolutions of the brain arteries, convolutions that will prevent blood flow will occur in the membrane structure. When the main reasons for these effects were investigated, it was seen that the shape of the narrowing through which the fluids pass could have an impact on the flow profile, and that important parameters such as the vacuum power at the narrowing outflow section and the speed of the linear flow were strongly related to the flow profile [15-17].

In the treatment of aneurysms that have ruptured or require intervention, it is not possible to drain the blood in the subarachnoid space with the current treatment methods or stents. Due to negativities such as fusiform and dissection type brain aneurysms being difficult to treat with said existing methods, and these treatment methods or stents causing mortality and morbidity due to rebleeding of the aneurysm after treatment, it has become necessary to provide a stent that removes the blood in the subarachnoid space, prevents the aneurysm from bleeding again, ensures the continuity of the arterial blood circulation distal to the aneurysm, reduces mortality and morbidity due to aneurysmal subarachnoid haemorrhage in the early and late stages of aneurysmal subarachnoid haemorrhage and is suitable for different vessel shapes or diameters, for use in the treatment of aneurysms that have ruptured or require intervention in the relevant field.

### BRIEF DESCRIPTION AND AIMS OF THE INVENTION

The invention describes a stent that reduces damage after brain haemorrhage, for use in the treatment of aneurysms that have ruptured or require intervention. The stent of the invention comprises an exoskeleton, a curved conical structure positioned in the centre of the stent, bridge, flap and silicone sheath.

The most important aim of the invention is to provide a stent for use in the treatment of brain aneurysms that have ruptured or require intervention. The invention comprises a curved conical structure and flaps. By means of this curved-conical structure, blood flow is accelerated in accordance with the conservation of energy (Bernoulli principle) and continuity equation. By increasing the energy converted into kinetic energy by means of the flap, blood pressure decreases and blood flow rate increases. In addition, the curved conical structure and flap structures increase the stability of the jet stream bundle at the outflow section, preventing turbulence formations that may occur in the area where blood will be absorbed at lumen area narrowing rates of 50% and above and disrupt the absorption of blood from the subarachnoid space into the vessel. Another aim of the invention is to provide a stent that drains the blood accumulated in the subarachnoid area due to aneurysm rupture, for use in the treatment of brain aneurysms that have ruptured or require intervention. By means of its curved-conical structure, flap and bridge, the stent that is the subject of the invention uses Bernoulli and flow continuity principles to reduce the pressure at the outflow section and drains the blood from the subarachnoid space with the vacuum effect. The curved conical structure increases the blood flow rate and reduces the pressure by narrowing the lumen area. Since the flap rotates the blood and extends its path, it provides additional energy loss and, with the effect of radial contraction, moves the region where maximum speed occurs beyond the outflow section. Additionally, the flap structure increases the vacuum power with the mixing effect it creates. The bridge, on the other hand, uses the pulse wave effect and produces an additional vacuum power, ensuring the absorption of blood from the subarachnoid space. Thus, with its curved-conical structure, flap and bridge elements, the designed stent enables the evacuation of blood accumulated in the subarachnoid area due to aneurysm rupture.

Another aim of the invention is to provide a stent that prevents rebleeding of the aneurysm, for use in the treatment of aneurysms that have ruptured or require intervention. The stent of the invention prevents blood from exiting into the subarachnoid space by changing the flow in the vessel in a way that creates a vacuum space under the aneurysm neck, by means of its curved-conical structure, flap and bridge. Thus, rebleeding of the aneurysm is prevented with the stent of the invention.

Another aim of the invention is to provide a stent that maintains the continuity of arterial blood circulation distal to the aneurysm by ensuring continuity of blood flow during treatment, for use in the treatment of aneurysms that have ruptured or require intervention. Since the curved conical structure inside said stent will maintain the amount of blood flowing per unit time at the outflow section according to the continuity equation by means of its structure that narrows the lumen area, according to the law of conservation of energy, it increases the flow rate of the blood and decreases its pressure. In addition, the vacuum created at the outflow section by means of the curved conical structure and flap prevents blood from exiting into the subarachnoid space, thus ensuring the continuity of blood circulation.

Another aim of the invention is to provide a stent that reduces mortality and morbidity due to aneurysmal subarachnoid haemorrhage in the early and late stages of aneurysmal subarachnoid hemorrhage, for use in the treatment of aneurysms that have ruptured or require intervention. Since the stent of the invention reduces the amount of blood filling the subarachnoid space during bleeding, the symptoms occurring in the early and advanced stages are improved with this stent.

Another aim of the invention is to provide a stent that accords with different vessel shapes and diameters for use in the treatment of aneurysms that are ruptured or require intervention. Said accord is achieved by scaling the outer body and conical narrowing of the stent to be used according to the anatomy and diameter of the vessel where the aneurysm is located, and by preserving the characteristic features of the lumen area narrowing, curved conical structure and flaps. The length of the bridge and silicone sheath is decided by taking into account the diameter of the aneurysm neck. The length of the bridge varies depending on the orientation angle. Orientation angle is the angle that the elements forming the bridge structure make with the direction of blood flow. In order to prevent the flow from remaining within the vascular lumen and directing into the aneurysm structure, the silicone sheath fixed to the stent body on the inflow section side covers a part of the aneurysm neck. Thus, the stent adapts to different vessel shapes and diameters.

Another aim of the invention is to provide a stent for use in the treatment of aneurysms that are ruptured or require intervention, facilitating the use of anticoagulant, thrombolytic and antiplatelet drugs in the early stages of treatment. The stent that is the subject of the invention comprises a curved-conical structure and at least one flap and directs the blood flow in the relevant vessel appropriately. The curved conical structure and flap change the blood flow. In addition, the bridge located under the neck of the aneurysm is designed to have a low metal-to-tissue ratio to prevent blood absorption from the subarachnoid space. The metal-to-tissue ratio of the stent is calculated by dividing the total amount of metal by the area of the vessel covered by the stent. By ensuring continuity of flow and facilitating the absorption of blood from the subarachnoid space, the stent of the invention prevents clot formation that may embolise into the distal circulation, which occurs through mechanisms such as angiographic vasospasm caused by bleeding due to aneurysm rupture, disorders in microcirculatory circulation, microthrombus formation and coagulation of blood in the subarachnoid space. The stent of the invention achieves this effect by facilitating the use of anticoagulant, thrombolytic and antiplatelet drugs, as it changes the blood flow to remain within the vessel.

With the invention, a stent that allows the drainage of blood accumulated in the subarachnoid area due to aneurysm rupture, prevents the aneurysm from bleeding again, maintains arterial circulation distal to the aneurysm by ensuring continuity of blood flow during treatment, reduces mortality and morbidity due to aneurysmal subarachnoid haemorrhage in the early and late stages of aneurysmal subarachnoid haemorrhage, adapts to different vessel shapes and diameters and facilitates the use of anticoagulant, thrombolytic and antiplatelet drugs in the early stages of treatment by appropriately directing the blood flow within the relevant vessel, for use in the treatment of aneurysms that have ruptured or require intervention.

### DESCRIPTION OF DRAWINGS

FIG. 1. Side view of the stent.

FIG. 2. View of the stent separated in the middle.

FIG. 3. Rear isometric view of the stent.

FIG. 4. Background view of the stent.

FIG. 5. Isometric view of the curved conical structure and silicone sheath.

FIG. 6. Side view of the curved conical structure and silicone sheath.

FIG. 7. Demonstration of the use of the stent in ruptured aneurysms.

FIG. 8. Demonstration of the use of the stent in fusiform aneurysms.

FIG. 9. Demonstration of the function of tearing the curved-conical structure from areas weakened by balloon angioplasty.

FIG. 10. The appearance of the curved-conical structure after rupture with the balloon angioplasty method.

FIG. 11. Situation after the placement of the second intracranial stent structure, following the tear function.

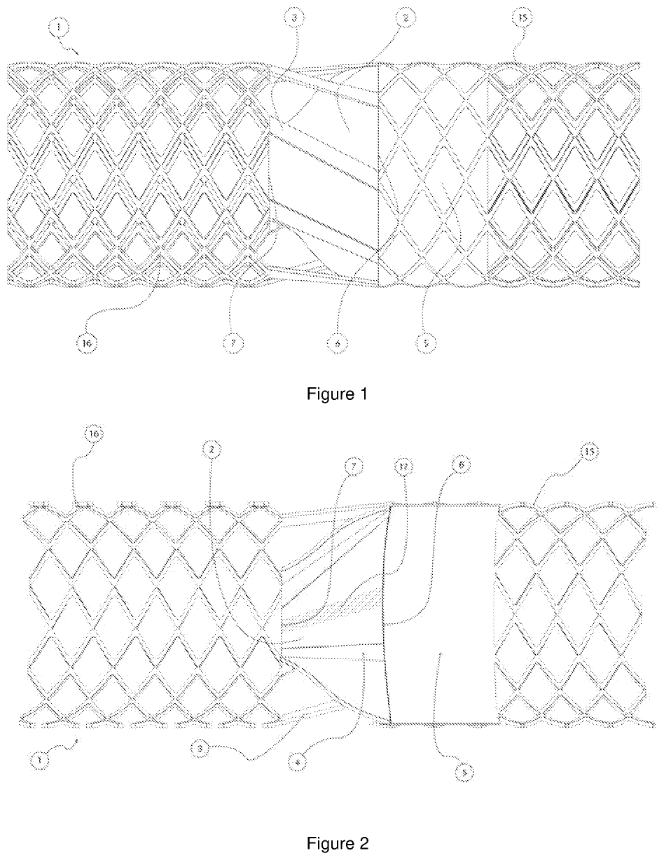

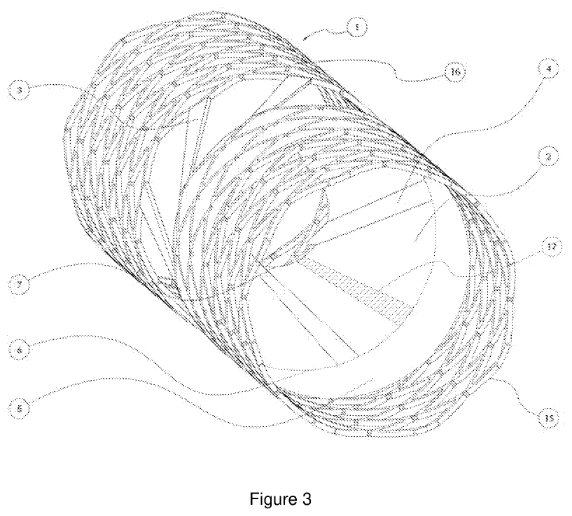

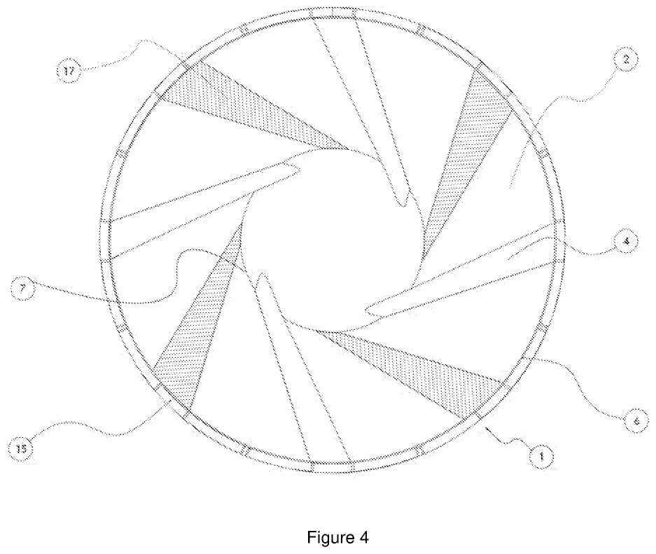

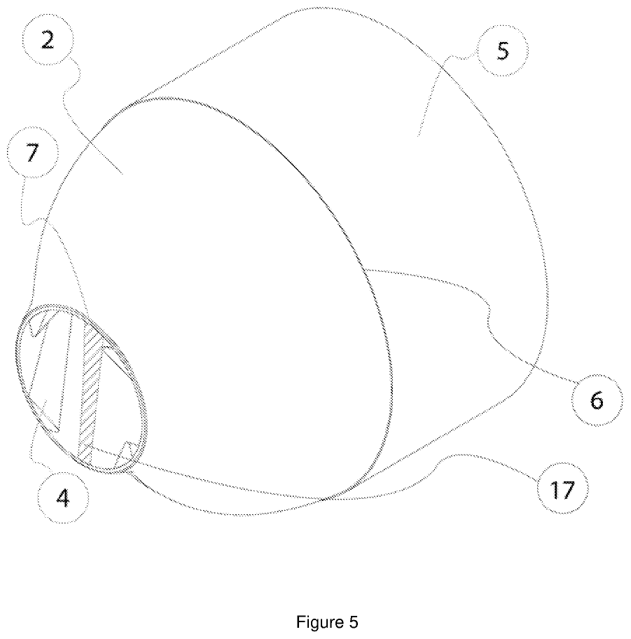

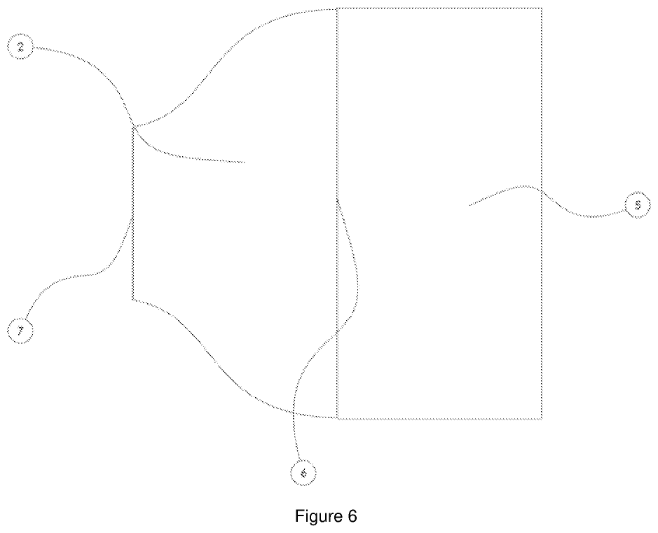

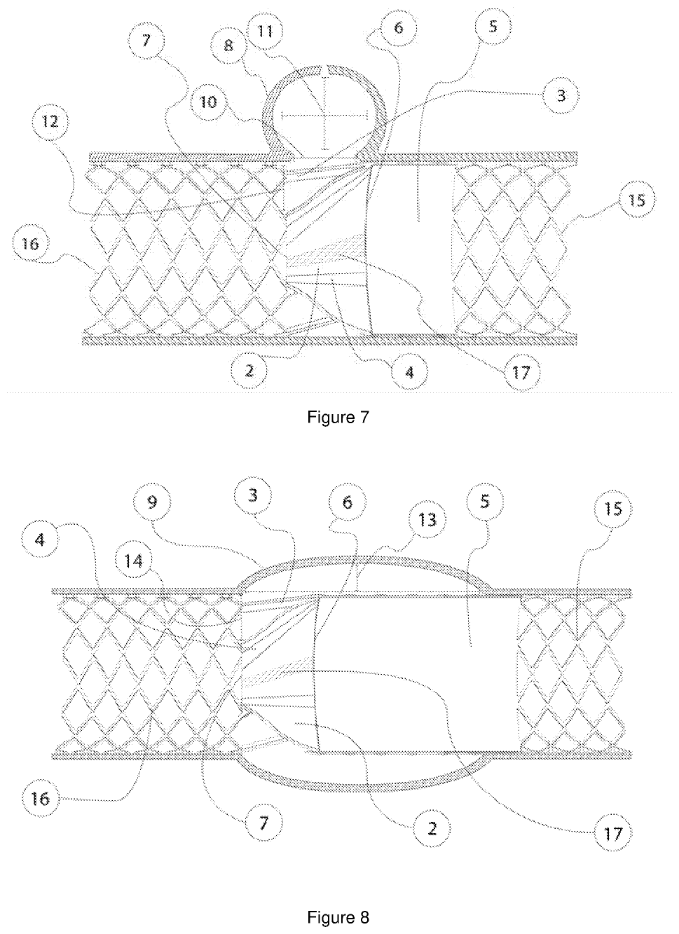

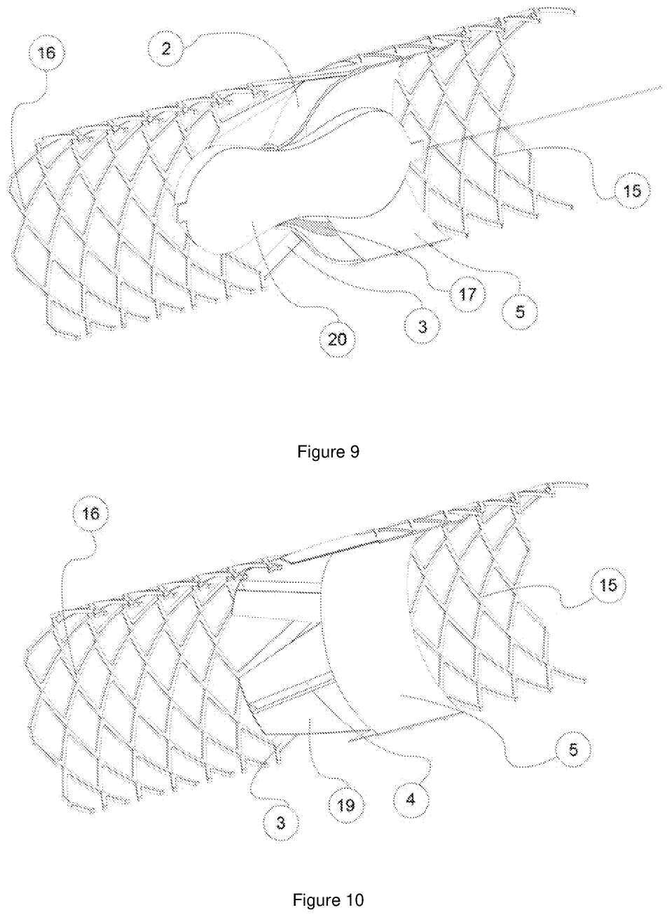

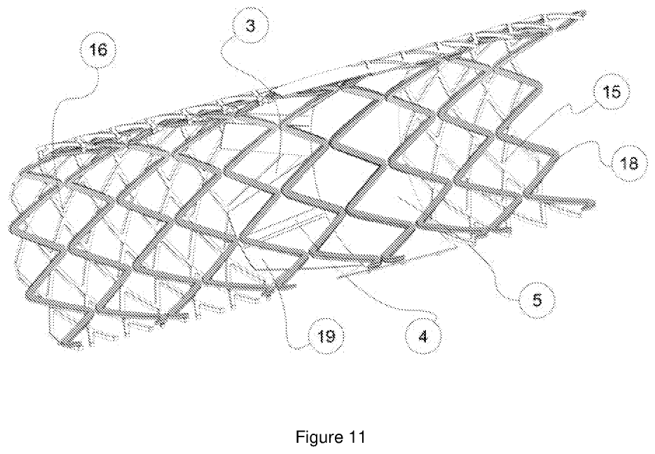

### DESCRIPTIONS OF REFERENCE NUMBERS IN DRAWINGS

1. Exoskeleton
2. Curved conical structure
3. Bridge
4. Flap
5. Silicone sheath
6. Inflow section
7. Outflow section
8. Sac-shaped (saccular) aneurysm
9. Fusiform aneurysm
10. Aneurysm neck
11. Centre of saccular aneurysm
12. Distal border of the aneurysm neck
13. Centre of fusiform aneurysm
14. Distal border of the fusiform aneurysm
15. Inflow section side stent body
16. Outflow section side stent body
17. Weakened area
18. Second intracranial stent structure
19. Leaf form
20. Balloon catheter

### DETAILED DESCRIPTION OF THE INVENTION

The invention relates to a stent that reduces the damage that occurs after brain haemorrhage, for use in the treatment of aneurysms that have ruptured or require intervention. In the invention, a stent that uses blood flow as vacuum power through an endovascular approach in the acute phase and thus removes the blood in the subarachnoid space is provided. The aneurysm mentioned here is saccular aneurysm (8), fusiform aneurysm (9) or dissection type brain aneurysm.

The stent that is the subject of the invention comprises
An exoskeleton (1) that carries the curved conical structure (2) at its centre in order to place the curved conical structure (2) under the aneurysm neck (10), forms the general structure of the stent with the inflow section side stent body (15), bridge (3) and outflow section side stent body (16) parts and ensures the continuity of the flow by preventing the narrowing of the relevant vessel as a result of vasoconstriction due to aneurysm rupture,
A curved conical structure (2) consisting of a flexible silicone membrane that allows the blood that creates vacuum force to be evacuated from the subarachnoid space by using Bernoulli's principle and the principle of continuity of flow,
A bridge (3), which ensures the integrity of the exoskeleton (1) structure by counteracting the mechanical stress on the exoskeleton (1) structure, created by the rotation of the flap (4) structures to the blood flow, and contributes to the evacuation of blood from the subarachnoid space with the pulse wave effect,
At least one flap (4), which protects the curved conical structure (2) in the centre of the stent, and the form of the inlet (6) and outlet (7) and provides rotation to the blood flow, and
Silicone sheath (5) that is fixed to the inflow section side stent body (15) and ensures leak-free flow of blood within the stent.

Said exoskeleton (1) is made of nickel-titanium (nitinol) alloy. The exoskeleton (1) structure is formed by the combination of the inflow section side stent body (15), outflow section side stent body (16) and bridge (3). The exoskeleton (1) fits snugly to the vessel, opens the vessel collapsed due to bleeding, and provides support for the neck of the aneurysm (10). In addition, the exoskeleton (1) comprises the curved conical structure (2) fixed inside it, and this curved conical structure (2) is aligned under the aneurysm neck (10) when placing the stent. The exoskeleton (1) constitutes the main structure of the stent and ensures continuity of flow by preventing narrowing of the relevant vessel by vasoconstriction due to aneurysm rupture.

The curved conical structure (2) provides narrowing of the lumen area with its sigmoidal shape. The lumen area narrowing rate is calculated by Formula I.

$$[ ( 1 - \frac{O}{I} ) \times 100 ] \qquad Formula I$$

O: Outflow section (7) area,
I: Inflow section (6) area.

Since the curved conical structure (2) is designed to benefit from Bernoulli and flow continuity principles, it creates a vacuum force with a pressure drop and speed increase at the outflow section (7), allowing the blood to be evacuated from the subarachnoid space. In addition, the curved conical structure (2) changes the blood flow leaking into the subarachnoid space in a way that creates a vacuum and draws it into the vessel, thus preventing rebleeding in the relevant aneurysm. Said curved conical structure (2) is beneficial in improving the clinical course when used with intrathecal fibrinolytic drugs. The curved conical structure (2) increases the stability and vacuum force of the jet stream by means of its sigmoidal shape. In addition, the curved conical structure (2) prevents the formation of potential vortices by making the current beam at the outflow section (7) more collected. The outflow section (7) is marked with a radiopaque material to facilitate the application of the stent. The curved conical structure (2) is resistant to pressure by means of its flexible structure, and the weakened areas (17) can only be torn with balloon angioplasty. In this way, the vascular lumen can be restored to its previous state when there is no longer a need for lumen narrowing. After the balloon angioplasty procedure, the flow-directing stent effect can be achieved by increasing the metal-tissue ratio with the second intracranial stent structure (18) to be placed inside the exoskeleton (1) structure. In addition to all these, with the placement of the second intracranial stent (18), the torn curved-conical structure (2) parts are opened in the form of a leaf (19). At the end of this process, the leaf forms (19) will remain between the second intracranial stent structure (18) placed inside and the exoskeleton (1) structure, thus serving as a barrier to reduce the blood flow under the aneurysm neck (10).

The flap (4) that is the subject of the invention is made of silicone material. Since this flap (4) adds rotation to the blood flow and causes kinetic energy loss, it provides an additional pressure drop at the outflow section (7) and therefore an increase in vacuum force. Rotation ensures that the outflow section (7) flow remains stable, and the flow bundle is distributed beyond the area where blood is absorbed below the aneurysm neck (10). Dispersing the flow beam away from the vacuum field will increase the vacuum force due to the mixing effect. In addition, the flap (4) prevents the water hammer effect caused by the contraction that may create a vacuum effect. Water hammer effect is the potential vibration that may occur proximal to the interruption due to the sudden interruption of the laminar flow due to narrowing. This vibration can create additional stress on arteries that are already inflamed and prone to vasoconstriction, triggering narrowing. The flap (4), on the other hand, prevents the water hammer effect as it prevents the sudden interruption of blood flow by extending the path of the blood with the rotation it gives to the blood. The flap (4), which is the subject of the invention, is a specially shaped ridge and adds general strength to the curved-conical structure (2), as well as increasing the energy converted into kinetic energy thanks to the rotational flow it gives to the blood. Thus, by taking advantage of Bernoulli's principle, it increases the potential energy loss and provides an additional increase in vacuum power. In addition, the flap (4), with the radial contraction effect provided by the rotation it gives to the blood, increases the distance over which the pressure drop at the outflow section (7) continues and reduces the turbulence effects that may be caused by the narrowing, allowing the blood absorbed from the subarachnoid space to be included in the flow more easily. With the current pressure drop at the outflow section (7), the risk of rupture is reduced by reducing the pressure on the vessel wall in unruptured aneurysms. The number of flaps (4) is calculated by dividing the circumference of the circular section, which will be calculated according to the diameter of the vessel to which the stent will be applied, by the width of the flap (4) and the sum of the spaces between the flaps (4). The number of flaps (4) is calculated by Formula II.

$$Kanatçik ( 4 ) sayisi = \frac{2 π r}{W + GW} \qquad Formula II$$

wherein:
r: The diameter of the vessel to which the stent will be applied
W: The width of the flap (4)
GW: The width of the gap between the flaps (4).

The silicone sheath (5) is a cylindrical structure made of silicone material and fixed to the inflow section stent body (15) of the nitinol exoskeleton (1) in order to ensure that the blood flow reaches the inflow section (6) without leakage. In cases where the aneurysm neck (10) is wide (>4 mm), the silicone sheath (5) covers a part of the aneurysm neck (10) and ensures that blood is absorbed from the subarachnoid space through the open part of the neck above the outflow section (7). The length of the silicone sheath (5) is decided according to the dimensions of the aneurysm neck (10).

Since the silicone sheath (5), curved conical structure (2) and flap (4) elements are produced from silicone, which is a hypoallergenic, flexible and shape-maintaining material, they maintain their form when compressed for endovascular application of the stent.

By having the same orientation angle as the rotation of the blood, the bridge (3) structure protects the main stent body from torsional stress that may occur due to the rotational flow of blood, while it does not prevent the blood flow from the aneurysm neck (10) through the gaps that will occur in the vacuum field. By means of these gaps, the bridge (3) provides an additional increase in vacuum power with a milking-like movement, using the pulse wave effect. The pulse wave effect is the instantaneous increase and decrease in pressure that originates from the heart and spreads to the entire arterial system. This pressure change causes minimal changes in artery lumen diameter. These minimal changes are continuous periodic movements and provide milking-like peristaltic movement towards the direction of spread. Since the bridge (3) has a low metal-to-tissue ratio, these minimal movements in the vessel wall affect the lumen and contribute to the vacuum effect. The bridge (3) is made of nitinol alloy. The bridge (3) applies a force opposite to the direction of the rotational force given to the blood by the flap (4) within the curved conical structure (2). The rotation given to the blood by the flaps (4) and the extension of the bridges (3) must be in the same direction. Thus, the bridge (3) facilitates the evacuation of blood from the subarachnoid space by reducing the metal-tissue ratio in the area where blood will be absorbed, while maintaining the solidity of the general structure of the stent.

In an embodiment of the invention, if the aneurysm located in the vessel is a saccular aneurysm (8), the curved conical structure (2) in the stent that is the subject of the invention is placed under the centre of the saccular aneurysm (11), with its outflow section (7) corresponding to the distal border (12) of the aneurysm neck.

In another embodiment of the invention, if the aneurysm in the vein is a fusiform aneurysm (9), the curved conical structure (2) in the stent that is the subject of the invention is placed under the centre of the fusiform aneurysm (13), with its outflow section (7) at the distal border (14) of the fusiform aneurysm.

In another embodiment of the invention, in case the aneurysm located in the vessel is a dissection type brain aneurysm, the curved conical structure (2) in the stent that is the subject of the invention is placed under the centre of the dissection type brain aneurysm, with its outflow section (7) corresponding to the area where blood flow is active.

After the stent that is the subject of the invention is entered into the circulatory system by catheterisation from the femoral artery for saccular aneurysms (8), fusiform aneurysms (9) and dissection type aneurysms that are ruptured or that require intervention, it passes through the arcus aorta and common carotid artery, then passes through the internal carotid artery and reaches the cerebral circulation. Then, the area of the brain vessel with the aneurysm is accessed. The outflow section (7) of the stent that is the subject of the invention is placed to align with the distal border (12) of the ruptured aneurysm neck, to align with the distal border (14) of fusiform aneurysms, and to align with the area where blood flow is active in dissection type aneurysms. In cases where the narrowing created by the curved conical structure (2) must be eliminated after the blood is drained from the subarachnoid space or aneurysm structures and/or due to another reason as a result of treatment, with the balloon angioplasty method, the curved conical structure (2) is torn from its weakened areas (17) and the lumen diameter is equalised to the exoskeleton (1) diameter. After this process, the flow-directing stent effect can be created by increasing the metal-tissue ratio with the second intracranial stent (18) to be placed within the exoskeleton (1) structure of the relevant invention. In addition to all these, when the second intracranial stent (18) is placed, the parts of the conical structure (2) torn by balloon angioplasty will open in the form of a leaf (19) and remain between the second intracranial stent (18) placed inside and the exoskeleton (1) structure, serving as a barrier to reduce blood flow under the aneurysm neck (10).

In the stent that is the subject of the invention, the narrowing formed by the curved conical structure (2), silicone sheath (5) and flaps (4) is designed according to the Bernoulli principle of fluid dynamics, the continuity equation principle and the Venturi effect based on these principles. The ratio of the outflow section (7) cross-sectional area to the inflow section (6) cross-sectional area directly affects the narrowing in the lumen area. It is known that in order to achieve pressure drops within the vessel that will create a vacuum effect in the aneurysm area, the rate of lumen area narrowing must be 50% or more. Therefore, in the stent design, the ratio of the outflow section (7) cross-sectional area to the inflow section (6) cross-sectional area was ensured to be 50% or less. Meanwhile, the curved shape, which is of critical importance in preventing turbulence and water hammer effects, must also be preserved. The curved shape is a sigmoidal constriction shape created by adopting the constriction angle revealed by experimental studies. Since the rate of lumen area narrowing that will create a vacuum effect depends on variables such as the diameter and shape of the vessel to which it will be applied, the curved narrowing geometry must be found experimentally for various situations. For various lumen area narrowing, the narrowing angle is found by simulations. The effect created by the curved conical structure (2) with lumen narrowing is calculated using Bernoulli's Principle and Venturi Effect, as shown in Formula III.

$${P}_{1} + \frac{1}{2} ρ {v}_{1}^{2} + {ρgh}_{1} = {P}_{2} + \frac{1}{2} ρ {v}_{2}^{2} + {ρgh}_{2} \qquad Formula III$$

wherein:
ρ=Fluid density
g=Gravitational acceleration,
P₁=Pressure at first level,
v₁=Speed at first level,
h₁=Height at first level,
P₂=Pressure at second level,
V₂=Speed at second level,
h₂=Height at second level.

The curved conical structure (2) in the stent that is the subject of the invention preserves the amount of blood flowing per unit time at the outflow section (7) by means of its structure that narrows the flow cross-sectional area according to the continuity equation, and thus reduces the pressure while increasing the flow rate of the blood in accordance with the law of conservation of energy. The conical narrowing of the curved conical structure (2) in the stent reduces the risk of rupture by reducing the pressure on the vessel wall in unruptured aneurysms with the existing pressure drop at the outflow section (7). In ruptured aneurysms, this conical structure stops the bleeding outside the vessel with the effect of the pressure drop, and with the vacuum effect, takes the blood that has leaked into the extravascular area into the vessel and returns it to circulation. In addition, in accordance with Bernoulli's Principle, the kinetic energy spent on rotation with the effect of the flaps (4) provides an additional decrease in pressure energy and creates an additional increase in vacuum power. Since the mentioned decrease shows a positive correlation with the strength of the rotation, it varies with parameters such as the shape, number and orientation angle of the flaps (4). In order to demonstrate the effect of the flap (4) element on the vacuum force, a computational flow simulation was made showing the pressure measurements at the outflow sections (7) of the stent structures before and after the addition of 4 identical flaps (4) to the stent structure. Pressure measurements at the outflow section (7) of the stent before and after adding the flap (4) structure are shown in Table 1 and Table 2, respectively.

TABLE 1
Pressure measurements at the outflow section
(7) before adding the flap (4) structure

| Target | | Average | Minimum | Maximum |
|---|---|---|---|---|
| Name | Unit | Value | Value | Value |
| Minimum | [mm Hg] | 19.25 | 18.78 | 19.55 |
| Total | | | | |
| Pressure | | | | |
| Average | [mm Hg] | 21.13 | 20.88 | 21.26 |
| Total | | | | |
| Pressure | | | | |
| Maximum | [mm Hg] | 33.03 | 32.10 | 33.91 |
| Total | | | | |
| Pressure | | | | |

TABLE 2
Pressure measurements at the outflow section
(7) after adding the flap (4) structure

| Target | | Average | Minimum | Maximum |
|---|---|---|---|---|
| Name | Unit | Value | Value | Value |
| Minimum | [mm Hg] | 17.08 | 16.77 | 17.39 |
| Total | | | | |
| Pressure | | | | |
| Average | [mm Hg] | 17.98 | 17.69 | 18.28 |
| Total | | | | |
| Pressure | | | | |
| Maximum | [mm Hg] | 27.03 | 26.79 | 27.26 |
| Total | | | | |
| Pressure | | | | |

TABLE 3
The change in the pressure values at the outflow section
(7) before and after adding the flap (4) structure

| | Change in | Change in | Change in |
|---|---|---|---|
| Compared | Average Value | Minimum | Maximum |
| Parameters | (%) | Value (%) | Value (%) |
| Minimum Total | −11.25 | −10.67 | −11.02 |
| Pressure | | | |
| Average Total | −14.89 | −15.27 | −14.02 |
| Pressure | | | |
| Maximum Total | −18.17 | −16.53 | −19.61 |
| Pressure | | | |

As shown in Table 3, when the minimum total pressure value, average total pressure value and maximum total pressure value in Tables 1 and 2 are compared, a decrease between 10.67% and 19.61% is observed in the pressure values at the outflow section (7) when the flap (4) is added. Therefore, the flap (4) structures contribute to the vacuum power and increase blood absorption from the subarachnoid space.

### REFERENCES

[1] Karhunen, V., Bakker, M. K., Ruigrok, Y. M., Gill, D., & Larsson, S. C. (2021). Modifiable Risk Factors for Intracranial Aneurysm and Aneurysmal Subarachnoid Hemorrhage: A Mendelian Randomization Study. Journal of the American Heart Association, 10 (22), e022277. https://doi.org/doi: 10.1161/JAHA.121.022277
[2] Toth, G., & Cerejo, R. (2018). Intracranial aneurysms: Review of current science and management. Vascular Medicine, 23 (3), 276-288. https://doi.org/10.1177/1358863x18754693
[3] Macdonald, R. L. (2014). Delayed neurological deterioration after subarachnoid haemorrhage. Nat Rev Neurol, 10 (1), 44-58. https://doi.org/10.1038/nrneurol.2013.246
[4] Reis, C., Ho, W. M., Akyol, O., Chen, S., Applegate, R., & Zhang, J. (2017). Chapter 25-Pathophysiology of Subarachnoid Hemorrhage, Early Brain Injury, and Delayed Cerebral Ischemia. In L. R. Caplan, J. Biller, M. C. Leary, E. H. Lo, A. J. Thomas, M. Yenari, & J. H. Zhang (Eds.), Primer on Cerebrovascular Diseases (Second Edition) (pp 125-130). Academic Press. https://doi.org/https://doi.org/10.1016/B978-0-12-803058-5.00025-4
[5] Aggarwal, A., Dhandapani, S., Praneeth, K., Sodhi, H. B. S., Pal, S. S., Gaudihalli, S., Khandelwal, N., Mukherjee, K. K., Tewari, M. K., Gupta, S. K., & Mathuriya, S. N. (2018). Comparative evaluation of H&H and WFNS grading scales with modified H&H (sans systemic disease): A study on 1000 patients with subarachnoid hemorrhage. Neurosurg Rev, 41 (1), 241-247. https://doi.org/10.1007/s10143-017-0843-y
[6] Florez-Perdomo, W., Mishra, R., García-Ballestas, E., Lozada-Martínez, I. D., Quiñones-Ossa, G. A., Joaquim, A., Agrawal, A., Janjua, T., Rahman, S., Suarez-Causado, A., & Moscote-Salazar, L. R. (2022). Cisternal irrigation and clot removal to prevent vasospasm and poor outcome in aneurysmal subarachnoid hemorrhage: Systematic review and meta-analysis. International Journal of Surgery Open, 43, 100459. https://doi.org/https://doi.org/10.1016/j.ijso.2022.100459
[7] Lu, X., Ji, C., Wu, J., You, W., Wang, W., Wang, Z., & Chen, G. (2019). Intrathecal Fibrinolysis for Aneurysmal Subarachnoid Hemorrhage: Evidence From Randomized Controlled Trials and Cohort Studies [Systematic Review]. Frontiers in Neurology, 10. https://doi.org/10.3389/fneur.2019.00885
[8] Aldrich, E. F., Higashida, R., Hmissi, A., Le, E. J., Macdonald, R. L., Marr, A., Mayer, S. A., Roux, S., & Bruder, N. (2020). Thick and diffuse cisternal clot independently predicts vasospasm-related morbidity and poor outcome after aneurysmal subarachnoid hemorrhage. J Neurosurg, 134 (5), 1553-1561. https://doi.org/10.3171/2020.3.Jns193400
[9] Shimamura, N., Fumoto, T., Naraoka, M., Katagai, T., Fujiwara, N., Katayama, K., Kinoshita, S., Yanagiya, K., Sasaki, T., Kurose, A., & Ohkuma, H. (2021). Irreversible Neuronal Damage Begins Just After Aneurysm Rupture in Poor-Grade Subarachnoid Hemorrhage Patients. Transl Stroke Res, 12 (5), 785-790. https://doi.org/10.1007/s12975-020-00875-0
[10] Chen, Y., Galea, I., Macdonald, R. L., Wong, G. K. C., & Zhang, J. H. (2022). Rethinking the initial changes in subarachnoid haemorrhage: Focusing on real-time metabolism during early brain injury. eBioMedicine, 83. https://doi.org/10.1016/j.ebiom.2022.104223
[11] Musmar, B., Adeeb, N., Ansari, J., Sharma, P., & Cuellar, H. H. (2022). Endovascular Management of Hemorrhagic Stroke. Biomedicines, 10 (1), 100. https://www.mdpi.com/2227-9059/10/1/100 Ten Brinck, M. F. M., Shimanskaya, V. E., Aquarius, R., Bartels, R., Meijer, F. J. A.,
[12] Koopmans, P. C., de Jong, G., Wakhloo, A. K., de Vries, J., & Boogaarts, H. D. (2022). Outcomes after Flow Diverter Treatment in Subarachnoid Hemorrhage: A Meta-Analysis and Development of a Clinical Prediction Model (OUTFLOW). Brain Sci, 12 (3). https://doi.org/10.3390/brainsci12030394
[13] Dholakia, R., Sadasivan, C., Fiorella, D. J., Woo, H. H., & Lieber, B. B. (2017). Hemodynamics of Flow Diverters. J Biomech Eng, 139 (2). https://doi.org/10.1115/1.4034932 [14] Chen, Z., Qin, H., Liu, J., Wu, B., Cheng, Z., Jiang, Y., Liu, L., Jing, L., Leng, X., Jing, J., Wang, Y., & Wang, Y. (2020). Characteristics of Wall Shear Stress and Pressure of Intracranial Atherosclerosis Analyzed by a Computational Fluid Dynamics Model: A Pilot Study [Original Research]. Frontiers in Neurology, 10. https://doi.org/10.3389/fneur.2019.01372
[15] Im, K. S., Cheong, S. K., Powell, C. F., Lai, M. C., & Wang, J. (2013). Unraveling the geometry dependence of in-nozzle cavitation in high-pressure injectors. Sci Rep, 3, 2067. https://doi.org/10.1038/srep02067
[16] Samad, A, Omar, R, Hewakandamby, B, Lowndes, I, & Short, G. “Swirl Induced Flow Through a Venturi-Ejector.” Proceedings of the ASME 2012 Fluids Engineering Division Summer Meeting collocated with the ASME 2012 Heat Transfer Summer Conference and the ASME 2012 10th International Conference on Nanochannels, Microchannels, and Minichannels. Volume 2: Fora. Rio Grande, Puerto Rico, USA. Jul. 8-12, 2012. pp. 65-70. ASME. https://doi.org/10.1115/FEDSM2012-72093
[17] Borynyak, K., Hrebtov, M., Bobrov, M., & Kozyulin, N. (2018). Numerical simulation of a low-swirl impinging jet with a rotating convergent nozzle. Journal of Physics: Conference Series, 980, 012016. https://doi.org/10.1088/1742-6596/980/1/012016

1. A stent that allows the drainage of blood accumulated in the subarachnoid area due to aneurysm rupture, prevents the aneurysm from bleeding again, maintains blood circulation distal to the aneurysm by ensuring continuity of arterial blood flow during treatment, reduces mortality and morbidity due to aneurysmal subarachnoid haemorrhage in the early and late stages of aneurysmal subarachnoid haemorrhage, adapts to different vessel shapes and diameters and facilitates the use of anticoagulant, thrombolytic and antiplatelet drugs in the early stages of treatment by appropriately directing the blood flow within the relevant vessel, comprising:
An exoskeleton (1) that carries the curved conical structure (2) at its centre in order to place the curved conical structure (2) under the aneurysm neck (10), forms the general structure of the stent with the inflow section side stent body (15), bridge (3) and outflow section side stent body (16) parts and ensures the continuity of the flow by preventing the narrowing of the relevant vessel as a result of vasoconstriction due to aneurysm rupture,
A curved conical structure (2) consisting of a flexible silicone membrane that allows the blood that creates vacuum force to be evacuated from the subarachnoid space by using Bernoulli's principle and the principle of continuity of flow,
A bridge (3), which ensures the integrity of the exoskeleton (1) structure by counteracting the mechanical stress on the exoskeleton (1) structure, created by the rotation of the flap (4) structures to the blood flow, and contributes to the evacuation of blood from the subarachnoid space with the pulse wave effect,
At least one flap (4), which protects the curved conical structure (2) in the centre of the stent, and the form of the inlet (6) and outlet (7) and provides rotation to the blood flow, and
Silicone sheath (5) that is fixed to the inflow section side stent body (15) and ensures leak-free flow of blood within the stent.

2. A stent according to claim 1, wherein said aneurysm is a ruptured or saccular aneurysm (8), a fusiform aneurysm (9) or a dissection type brain aneurysm that requires intervention.

3. A stent according to claim 1, wherein said exoskeleton (1) is made of nickel-titanium (nitinol) alloy.

4. A stent according to claim 1, wherein said bridge (3) is made of nitinol alloy.

5. A stent according to claim 1, wherein the number of said flaps (4) is calculated according to Formula I below:
$$[ [ Kanatçik ( 4 ) sayisi = \frac{2 π r}{G + B} ] ] \qquad Formula I$$
$$Number of flaps ( 4 ) = \frac{2 π r}{G + B}$$
wherein
r: The diameter of the vessel to which the stent will be applied
W: The width of the flap (4)
GW: The width of the gap between the flaps (4).

6. A stent according to claim 1 for use in the treatment of saccular aneurysm (8), fusiform aneurysm (9) or dissecting type brain aneurysm that has ruptured or requires intervention.
